(19) United States
(12) Patent Application Publication
(10) Pub. No.: US 2018/0055882 A1
(43) Pub. Date: Mar. 1, 2018

(54) FUNCTIONAL IMMUNOPHENOTYPING OF LEUKOCYTES IN HUMAN TISSUES

(71) Applicant: The Regents of the University of California, Oakland, CA (US)

(72) Inventors: Michael Rosenblum, San Francisco, CA (US); Adil Daud, Hillsborough, CA (US); Mariela Pauli, San Francisco, CA (US); Margaret Lowe, San Francisco, CA (US)

(21) Appl. No.: 15/666,199

(22) Filed: Aug. 1, 2017

Related U.S. Application Data
(60) Provisional application No. 62/370,997, filed on Aug. 4, 2016.

(51) Int. Cl. A61K 35/17; A61K 39/395; C07K 16/28; G01N 33/569

(52) U.S. Cl. CPC A61K 35/17; A61K 39/39558; A61K 2039/505; G01N 33/56972; C07K 16/2818

(57) ABSTRACT

Methods of detecting surface markers on T-cells from skin samples are provided. T-cells can be isolated from skin samples for example using single density centrifugation. The percentage of CD8⁺ cells being CTLA4⁺PD-1⁺ can be used to predict responsiveness to anti-PD-1 therapy.

### CROSS-REFERENCE TO RELATED PATENT APPLICATIONS

The present application claims benefit of priority to U.S. Provisional Patent Application No. 62/370,997, filed Aug. 4, 2016, which is incorporated by reference for all purposes.

### BACKGROUND OF THE INVENTION

Tumors employ multiple mechanisms to suppress adaptive immune responses directed at antigens expressed in the tumor microenvironment. In this context, chronic and persistent antigen stimulation results in increased expression of PD-1 on CD8⁺ T cells infiltrating neoplastic tissue (Pauken K E, Wherry E J., Trends Immunol. 2015; 36(4):265-276). The net result of signaling through this receptor is an attenuation of the cytotoxic and cytokine-producing capacity of these cells, leading to ineffective anti-tumor immune responses (Pauken K E, Wherry E J., Trends Immunol. 2015; 36(4):265-276). Targeted inhibition of the PD-1 pathway is showing excellent efficacy in several human tumors (Brahmer J R et al., J. Clin. Oncol. Off. J. Am. Soc. Clin. Oncol. 2010; 28(19):3167-3175; Hamid O et al., N. Engl. J. Med. 2013; 369(2):134-144; Rizvi N A et al., Lancet Oncol. 2015; 16(3):257-265); however, many patients do not respond, and the cellular and molecular mechanisms underlying this clinical heterogeneity are only beginning to be elucidated. A biomarker that accurately predicts clinical response to anti-PD-1 therapy is useful in order to appropriately select patients for this therapy in the face of multiple emerging treatment options for metastatic cancer.

It is becoming increasingly clear that the immune composition in tumors is markedly different from that observed in peripheral blood (Ahmadzadeh M et al., Blood 2009; 114(8):1537-1544; Gros A et al., J. Clin. Invest. 2014; 124(5):2246-2259). Quantification of PD-L1 and PD-1 expression in tumors by routine immunohistochemistry has been utilized in an attempt to predict response to anti-PD-1 therapy (Madore J et al., Pigment Cell Melanoma Res. 2015; 28(3):245-253; Mahoney K M, Atkins M B., Oncol. Williston Park N2014; 28 Suppl 3:39-48; Pan Z-K et al., J. Thorac. Dis. 2015; 7(3):462-470; Patel S P, Kurzrock R., Mol. Cancer Ther. 2015; 14(4):847-856; Tumeh P C et al., Nature 2014; 515(7528):568-571). These studies have yielded provocative results; however, constraints in the number of markers able to be simultaneously assessed and the inherent difficulties in quantifying staining intensity have limited the potential of this approach. In addition, although standard immunohistochemistry reveals information regarding which immune cells are present and where they localize within tumors, it rarely elucidates how these cells are functioning. In the current study, we utilize multi-parameter flow cytometry to comprehensively analyze the tumor immune microenvironment prior to anti-PD-1 therapy. Using this approach, we quantify the accumulation of a unique immune cell population that robustly predicts response to this treatment. In functional experiments, we show that this cell subset represents a partially exhausted tumor-infiltrating T cell and that treatment with anti-PD-1 effectively activates these cells in tumors.

### BRIEF SUMMARY OF THE INVENTION

In one aspect, a method of determining a percentage of CD8⁺ T-cells being CTLA4⁺PD-1⁺ in a skin sample comprising a metastatic lesion from a human individual is provided. In some embodiments, the method comprises:
digesting the skin sample in a digestion medium comprising a protease, a nuclease, or both a protease and a nuclease to generate a digested skin sample;
centrifuging the digested skin sample in a single density solution to separate epithelial cells, T-cells, and insoluble debris;
extracting the T-cells from the single density solution, thereby separating the lymphocytes from at least some of the epithelial cells and insoluble debris;
and contacting the extracted T-cells with: (a) an anti-CD8 antibody, (b) an anti-CTL4 antibody, and (c) an anti-PD-1 antibody and calculating the percentage of CD8+ T-cells being CTLA4⁺PD-1⁺ based upon the amount of anti-CD8 antibody, anti-CTL4 antibody, and anti-PD-1 antibody that bind the extracted T-cells.

In some embodiments, the method further comprises after the digesting and before the centrifuging, agitating the digested skin sample in a mixing solution. In some embodiments, the mixing solution comprises serum. In some embodiments, the mixing solution comprises fetal bovine serum, RPMI medium, and one or more antibiotic.

In some embodiments, the protease is a collagenase and the nuclease is a DNAse.

In some embodiments, the metastatic lesion is a melanoma metastatic lesion.

In some embodiments, the method further comprises comparing the percentage to a cut-off value, and optionally wherein if the percentage exceeds the cut-off value, the method further comprises administering a therapeutic amount of a PD-1 antibody to the individual. In some embodiments, the cut-off value is between 10-40% (e.g., 20-30%) CTLA4⁺PD-1⁻CD8⁺ T-cells.

In another aspect, a method is provided of converting a human individual having a metastatic lesion in skin and who is non-responsive to anti-PD-1 therapy into a responder to anti-PD-1 therapy. In some embodiments, the method comprises: obtaining CTLA4⁺PD-1⁺CD8⁺ cells from the individual; expanding the CTLA4⁻PD-1⁺CD8⁺ cells ex vivo; and introducing the expanded cells into the individual, thereby increasing the responsiveness of the individual to anti-PD-1 treatment. In some embodiments, the individual has melanoma. In some embodiments, the method further comprises administering an anti-PD-1 molecule to the individual after the introducing. In some embodiments, the anti-PD-1 molecule is an antibody.

In another aspect, a method of detecting a cell-surface marker in T-cells from a skin sample is provided. In some embodiments, the method comprises:
digesting the skin sample in a digestion medium comprising collagenase and DNAse to generate a digested skin sample;
centrifuging the mixed digested skin sample in a single density solution to separate epithelial cells, T-cells, and insoluble debris;
extracting the T-cells from the single density solution, thereby separating the lymphocytes from at least some of the epithelial cells and insoluble debris;
and detecting one or more cell surface markers on the extracted T-cells with an antibody, thereby detecting a cell-surface marker in T-cells from a skin sample.

In some embodiments, the protease is a collagenase and the nuclease is a DNAse.

In some embodiments, the method further comprises after the digesting and before the centrifuging, agitating the digested skin sample in a mixing solution. In some embodiments, the mixing solution comprises serum. In some embodiments, the mixing solution comprises fetal bovine serum, RPMI medium, and one or more antibiotic.

### BRIEF DESCRIPTION OF THE DRAWINGS

FIGS. 1A-1C. Relative abundance of CTLA-4⁺PD-1⁺ CTLs predicts response to anti-PD-1 therapy. (FIG. 1A) Flow cytometric data from metastatic tumors taken prior to anti-PD-1 therapy and representative pre-treatment and post-treatment computed tomography images from a patient that achieved a response (responder) and did not achieve a response (non-responder). Flow cytometric plots are pre-gated on live CD45⁺CD3⁺CD8⁺ cells. (FIG. 1B) Discovery cohort (n=20 patients) and (FIG. 1C) validation cohort (n=20 patients) of progression free survival (PFS) in patients that had ≧20% (dotted line) or ≦20% (solid line) tumor-infiltrating CTLA-4⁺PD-1⁺ CTLs. Statistical significance was determined by Log-Rank test.

FIG. 2. Clinical characteristics and CTL profiles of patients with metastatic melanoma that responded or did not respond to anti-PD-1 therapy. Histogram showing flow cytometric quantification of percentages of CTLA4⁺PD-1⁺ tumor-infiltrating T cells in the CD8⁺ CTL gate versus individual patient characteristics. Responders included patients with tumor target lesions that met RECIST 1.1 criteria for complete response (>99% reduction in the target lesions) or partial response (≧30% reduction in target lesions). Non-responders included patients with tumor target lesions that met RECIST 1.1 criteria for progression (≧20% increase in the target lesions) or stable disease (<30% reduction or <20% increase in tumor target lesions). n=40 patients. Tregs were defined as Foxp3^{+hi}CTLA-4⁺ cells within the live CD45+CD3+CD4+ gate. V, validation; D, discovery; I, ipilimumab; T, Targeted therapy with BRAF/MEK inhibitors; Liv, liver; LN, lymph node; S, Skin; Ova, Ovary.

FIGS. 3A-3B. Tumor-infiltrating CTLA-4⁺PD-1⁺CTLs have a partially exhausted phenotype. (FIG. 3A) Representative flow cytometric plots of intracellular cytokine staining of tumor-infiltrating lymphocytes obtained from metastatic tumors taken prior to anti-PD-1 therapy. Pre-gated on live CD45⁺CD3⁺CD8⁺ cells. (FIG. 3B) Quantification of cytokine expressing cells (as measured by intracellular cytokine staining) obtained from multiple metastatic tumors taken prior to anti-PD-1 therapy. Symbols represent individual patients. Lines represent mean values. Combined data from 3 replicate experiments. **p<0.01 by unpaired 2 tailed Student's t test.

FIGS. 4A-4C. Anti-PD-1 treatment results in preferential activation of tumor-infiltrating CD8⁺CTLs. (FIG. 4A) Flow cytometric quantification of tumor-infiltrating CD4⁺ and CD8⁺ T cells obtained prior to (pre) or after beginning (post) and PD-1 therapy. (FIG. 4B) Representative flow cytometric plots and flow cytometric quantification of HLA-DR expression on tumor-infiltrating CD8⁺ T cells prior to beginning or after beginning and PD-1 therapy. (FIG. 4C) Flow cytometric quantification of tumor-infiltrating Tregs as well as Treg expression of CTLA-4 and HLA-DR prior to beginning or after beginning anti-PD-1 therapy. Symbols represent individual patients. Lines represent mean values. MFI, mean fluorescence intensity; ns, not significant; p-values obtained by unpaired 2 tailed Student's t test.

FIGS. 5A-5B. Flow cytometric gating strategy. (FIG. 5A) Gating strategy for isolation of CD4⁺ Teff cells (live CD45⁺CD3⁺CD4⁺Foxp3⁻), CD4⁺ Treg cells (live CD45⁺CD3⁺CD4⁺Foxp3⁺) and CD8⁺ CTLs (CD45⁺CD3⁺CD4⁻CD8⁺). (FIG. 5B) Representative flow cytometric plots to quantify CTLA-4, HLA-DR, PD-1, and PD-L1 expression on Teff cells, Treg cells and CTLs.

FIGS. 6A-6C. Characterization of CTLA-4 and PD-1 expression on tumor-infiltrating CTLs and correlation with clinical response to anti-PD-1 therapy. (FIG. 6A) Representative flow cytometric plots and flow cytometric quantification of CTLA-4 expression on tumor-infiltrating CD8⁺CTLs in patients that responded or did not respond to anti-PD-1 therapy. (FIG. 6B) Representative flow cytometric plots and flow cytometric quantification of PD-1 expression on CTLA-4^{neg} and CTLA-4^{pos} tumor-infiltrating CD8⁺CTLs. (FIG. 6C) Flow cytometric quantification of CD4+ T cells, CD8+ T cells, the CD4:CD8 ratio, and regulatory T cells (Foxp3+ cells) in patients that responded or did not respond to anti-PD-1 therapy. All flow cytometric data was obtained from metastatic tumors prior to beginning treatment with anti-PD-1. Plots in (a) and (b) are pre-gated on live CD45⁺CD3⁺CD8⁺ cells. Symbols represent individual patients. MFI, mean fluorescence intensity. Lines represent mean values and bars represent standard error of the mean. p-values obtained by unpaired Student's t test. ns, non-significant.

FIG. 7. Relative abundance of CTLA-4⁺PD-1⁺CTLs correlates with overall survival. Overall survival (OS) in patients that had ≧20% (dotted line) or ≦20% (solid line) tumor-infiltrating CTLA-4⁺PD-1⁺ CTLs. Statistical significance was determined by Log-Rank test. (n=40).

FIG. 8. Combined expression of CTLA-4 and PD-1 on tumor-infiltrating CD8⁺ T cells strongly correlates with clinical response to anti-PD-1 therapy. Representative flow cytometric plot of tumor-infiltrating CD8⁺ T cells dividing cells into CTLA-4⁺PD-1⁺ double positive cells, CTLA-4⁻PD-1⁺ single positive cells, CTLA-4⁺PD-1⁻ single positive cells, and CTLA-4⁻PD-1⁻ double negative cells. Scatter plots represent clinical response data based on the percentage of these 4 populations present within tumors prior to beginning anti-PD-1 monotherapy. Plot is pre-gated on live CD45⁺CD3⁺CD8⁺ cells. Symbols represent individual patients. Lines represent mean values and bars represent standard error of the mean. p-values obtained by unpaired Student's t test. ns, non-significant. n=40 patients.

FIG. 9. Immunohistochemical analysis of tumor-infiltrating CD8+ T cells. Metastatic melanoma tumor samples were evaluated from 21 patients using the method described by Tumeh et. al. (Nature 2011) for abundance of cells at the tumor invasive margin and overall cell density. Of 21 samples, 8 specimens (38.1%) were not able to be evaluated because the invasion margin could not be determined. Of the 13 evaluable remaining samples, 4 patients were responders and 7 were non-responders and 2 patients were lost to follow-up. Dashed line represents 1500 CD8 T cells/mm² at the invasive margin used to calculate predictive values. Lines represent mean values and bars represent standard error of the mean.

### DEFINITIONS

An “anti-PD-1 agent” as used herein refers to an antibody or other therapeutic drug that specifically binds to human Programmed Cell Death Protein 1 (PD-1) and blocks binding of PD-1's ligand, programmed death ligand 1 (PD-L1), to PD-1. A non-exhaustive list of anti-PD-1 agents and a discussion of PD-1 therapy is described in, e.g., Swaike, et al., Molecular Immunology, 67(2), Part A, pp. 4-17 (2015).

In the context of “CTLA4⁺” and “PD-1⁺” refers to an increased staining intensity by a target-specific antibody (e.g., anti-CTLA4 or anti-PD-1, respectively) as measured by FACS of at least 10% compared to isotype control antibody staining of the same cells or compared to binding of the target-specific antibody to an internal cell population that does not express the target (e.g., live CD45+CD3− cells). As used herein, “CTLA4⁺” and “CTLA4^{high}” are synonymous. As used herein, “PD-1⁺” and “PD-1^{high}” are synonymous.

A “single density solution” as used herein in the context of centrifugation refers to a mixture in which the liquid portion has the same density throughout the mixture (after performing centrifugation non-solutes such as cells and insoluble debris will sort at different rates based in part on differences in density of the non-solutes). Single density solution centrifugation is different from density gradient centrifugation, in which different levels of the solution being centrifuged have different densities due to solubilization of different amounts of a solute, for example a polysaccharide, e.g., such a ficoll, or percoll, which is composed of colloidal silica coated with polyvinylpyrrolidone (PVP)).

The terms “therapy,” “treatment,” and “amelioration” refer to any reduction in the severity of cancer, including but not limited to a reduction in the number of tumor cells in an individual, a reduction in the number of metastases, or increased survival time. As used herein, the terms “treat” and “prevent” are not intended to be absolute terms. Treatment and prevention can be complete or partial. The effect of treatment can be compared to an individual or pool of individuals not receiving the treatment, or to the same patient prior to treatment or at a different time during treatment.

The terms “effective amount,” “effective dose,” “therapeutically effective amount,” etc. refer to that amount of the therapeutic agent sufficient to ameliorate or treat cancer, as described above. Therapeutic efficacy can also be expressed as “-fold” increase or decrease. For example, a therapeutically effective amount can have at least a 1.2-fold, 1.5-fold, 2-fold, 5-fold, or more effect over a control.

As used herein, the term “pharmaceutically acceptable” is used synonymously with physiologically acceptable and pharmacologically acceptable. A pharmaceutical composition will generally comprise agents for buffering and preservation in storage, and can include buffers and carriers for appropriate delivery, depending on the route of administration.

The terms “dose” and “dosage” are used interchangeably herein. A dose refers to the amount of active ingredient given to an individual at each administration The dose will vary depending on a number of factors, including frequency of administration; size and tolerance of the individual; type and severity of the condition; risk of side effects; and the route of administration. One of skill in the art will recognize that the dose can be modified depending on the above factors or based on therapeutic progress.

“Subject,” “patient,” “individual” and like terms are used interchangeably and refer to, except where indicated, humans. The term does not necessarily indicate that the subject has been diagnosed with a particular disease, but includes to an individual under medical supervision. A patient can be an individual that is seeking treatment, monitoring, adjustment or modification of an existing therapeutic regimen, etc.

### DETAILED DESCRIPTION OF THE INVENTION

### Introduction

The inventors have discovered new methods of predicting when melanoma patients will respond to anti-PD-1 treatment and have also discovered improved technical methods for assaying skin samples for the presence of biomarkers. For example, the inventors have discovered that human individuals that have a percentage of 30% or more CD8⁺ T-cells being CTLA4⁺PD-1⁺ in a skin sample comprising a metastatic melanoma lesion generally respond to anti-PD-1 therapy. Individuals that have a percentage of 20% or less CD8⁺ T-cells being CTLA4⁺PD-1⁺ in a skin sample comprising a metastatic melanoma lesion generally do not respond to anti-PD-1 therapy. Accordingly, one can predict an individual's responsiveness to anti-PD-1 treatment by determining the percentage of CTLA4⁺PD-1⁺CD8⁺ T-cells out of the total number of CD8⁺ T-cells in a skin sample from the individual. This allows for targeted treatments (e.g., with anti-PD-1 treatment) for individuals predicted to be responsive (and avoiding other treatments if unnecessary) and avoids the risks and side effects of anti-PD-1 treatment for those predicted unlikely to respond in favor of other treatments that may work for the individual.

Moreover, the inventors have discovered an improved method of detecting biomarkers in a skin sample that results in higher number of T-cells and fewer epithelial cells, allowing for greatly improved detection of target cells and therefore more accurate determination of percentage of cells having a particular biomarker. The improved method comprises centrifuging digested skin cells in a single density solution to separate T-cells from less dense epithelial cells and more dense cellular debris. This method has been found to be superior to, for example, filtration methods for separating cells.

### Methods of Obtaining Skin T-Cells

In some embodiments, a method of obtaining T-cells from a skin sample is provided. A skin sample can be obtained by biopsy. The skin sample can include a cancer lesion, which can be for example, a metastatic or primary melanoma skin lesion or a metastatic skin lesion from a non-melanoma primary cancer.

The sample can be of any appropriate size. In some embodiments, the skin samples comprises 3-10 mm² of skin, for example 4 mm² of skin. Subcutaneous fat is removed from the skin sample.

The skin sample can be digested with at least one protease or nuclease or, in some embodiments, both. To improve digestion, the skin sample can be cut, sliced, minced or otherwise physically treated to generate more surface area of the sample for contact to the enzyme(s). Exemplary proteases can include, but are not limited to, collagenases or dispases. A commercially-available enzyme useful in this context are the Liberase™ enzymes (Roche). Exemplary nucleases can include, but are not limited to, DNAse I. Digestion can be performed, for example, for 12 hours at 37° C. to generate single cell suspensions.

The digested samples can be harvested into a wash buffer optionally comprising serum, antibiotics, and/or a buffer and salts. In some embodiments, the serum is fetal bovine serum. In some embodiments, serum in RPMI medium is used as the wash buffer, optionally comprising one or more type of antibiotic to prevent growth of contaminants.

The sample cell suspension in wash buffer can then be agitated to further suspend any clumping cells. For example, the suspension can be agitated by inverting or shaking the suspension in a container such as a centrifugation tube. The resulting suspension can subsequently be centrifuged at speeds that do not clump the T-cells in the suspension, but is performed at a sufficient speed to separate cellular debris (that will clump), T-cells, and lighter epithelial cells that form a layer above the T-cells. For example, the cells can be centrifuged in 50 mL conical tubes at 100-500 RPM (e.g., 250-350 RPM) for 1-10 minutes, e.g., 4-6 minutes). In some embodiments, the cells are centrifuged as a liquid at a temperature below 20° C., for example at 4° C. In some embodiments, the cell suspension will be in a single density solution (in contrast to a density gradient centrifugation, in which different levels of the solution being centrifuged have different density due to solubilization of different amounts of a solute, for example a polysaccharide, e.g., such a ficoll, or percoll, which is composed of colloidal silica coated with polyvinylpyrrolidone (PVP).

Upon completion of centrifugation, the centrifuged container will include a top (including at least the surface) layer of cells that will include epithelial cells, a middle layer of cells that are enriched for T-cells from the sample, and a bottom layer of cellular debris. The middle layer can then be extracted from the container, thereby physically separating the layer enriched for T-cells from the epithelial cell layer and the cellular debris. Lymphocyte cell yields are enhanced by at least 4-fold using this methodology and at least a 3-fold enrichment is the target (i.e., CD45+CD3+ T cells) yield.

The resulting cells enriched for T-cells can then be analyzed for one or more biomarker(s), including but not limited to cell surface biomarkers. For example, the T-cells can be treated with detection reagents to detect markers on the T-cells. Any reagent(s) capable of specifically detecting a biomarker in the surface of the T-cells can be used. In some embodiments, the reagent can be or include one or more antibody, peptide, nucleic acid (including but not limited to an aptamer) or other reagent selected to specifically bind to a target biomarker. The reagents can be labeled with a detectable label. Examples of detectable labels include, but are not limited to, biotin, enzymes, radioisotopes, chemiluminescent labels, or fluorescent labels

In some embodiments, the cells from the middle layer are sorted based upon the presence, absence, or abundance or one or more cell surface marker. In some embodiments, the biomarkers used for positive or negative selection may be identified by immunoselection techniques that utilize antibodies including, but not limited to, fluorescence activated cell sorting (FACS), magnetic cell sorting, panning, and chromatography. Immunoselection of two or more markers on T-cells may be performed in one or more steps, wherein each step positively or negatively selects for one or more markers. When immunoselection of two or more markers is performed in one step using FACS, the two or more different antibodies may be labeled with different fluorophores.

Methods of sorting cells are well known to persons of ordinary skill in the art. Cell sorters generally are capable of separating a complex mixture of cells into fractions of a single cell type. Typically, the cells to be sorted are introduced as a thin jet of carrier liquid emanating from a small nozzle orifice. Shortly after leaving the nozzle, the fluid passes through the waist of one or more tightly focused laser beams. The scattered and fluorescence light from these interactions can be collected and analyzed to determine if there are events (e.g., the presence of a fluorescence signal indicating that a fluorophore-labeled monoclonal antibody is bound to the surface of a cell) that prompt the sorting of the cell by various means. More than one label can be monitored at a time. FACS (fluorescence activated cell sorters) can easily analyze cells at speeds greater than 200,000 events per second. Generally, the physics of the carrier fluid, however, and the statistics of distributing the cells among the droplets limits sort rates to about 50,000 cells per second. This combination of speed and reliable separation allows individual cells to be isolated for other uses, if desired.

As noted above, the inventors have discovered that the percentage of CD8+ T-cells that express CTLA4⁺PD-1⁺ can be used to predict responsiveness of an individual to anti-PD-1 treatment. Accordingly, in some embodiments, the middle layer of cells enriched for T-cells, as described above, is used to determine the percentage of CTLA4⁺PD-1⁺CD8+ T-cells in the sample out of the total number of CD8+ T-cells in the sample. In some embodiments, the percentage is determined by FACS analysis or other immunodetection or sorting methods, using anti-CTLA, anti-PD-1 and anti-CD8 antibodies or other specific binding reagents. Anti-CTLA, anti-PD-1 and anti-CD8 antibodies are readily available for purchase or can be readily generated as known in the art.

In some embodiments, following determination of the percentage of CTLA4⁺PD-1⁺CD8+ T-cells in the sample out of the total number of CD8+T-cells in the sample, the determined percentage can be compared to a cut-off value. For example, the inventors have found that individuals having over 30% CTLA4⁺PD-1⁺CD8+ T-cells out of the total number of CD8+ T-cells generally respond (Objective Response Rate by Response Evaluation Criteria in Solid Tumors (RECIST) >80%) to PD-1 treatment whereas individuals having less than 20% CTLA4^{hi}PD-1^{hi}CD8+ T-cells out of the total number of CD8+ T-cells generally do not respond to PD-1 treatment. Accordingly, a cut-off value can be set depending on desired sensitivity and specificity, for example at 10%, 20%, 30%, 40%, e.g., between 10-40%, and comparison of the determined percentage to the cut-off can be used guide treatment options.

In some embodiments, a medical treatment can be applied to the individual based at least partially on determination of percentage of CTLA4⁺PD-1⁺CD8+ T-cells in the sample. If the number of CTLA4⁺PD-1⁺CD8+ T-cells out of the total number of CD8+T-cells in the skin sample exceeds a cutoff value, then the individual can be treated with anti-PD-1 therapy. Anti-PD-1 therapy refers to administration of one or more types of molecules that bind to PD-1 and block binding of its ligand, programmed death ligand 1 (PD-L1). See, for example, Swaike, et al., Molecular Immunology, 67(2), Part A, pp. 4-17 (2015). In some embodiments, the anti-PD-1 agent is an antibody that binds PD-1. Pembrolizumab, Nivolumab, Durvalumab and Atezolizumab are exemplary monoclonal antibodies that target PD-1 and have established clinical activity. In other embodiments, the anti-PD-1 agent can be a small molecule, e.g., agents having a molecular weight of less than 1,500 daltons, and in some cases less than 1,000, 800, 600, 500, or 400 daltons.

Anti-PD-1 therapy can be administered alone or in combination with an anti-CTLA agent. A common anti-CTLA agent is ipilimumab. In some embodiments, if the number of CTLA4⁺PD-1⁺CD8+ T-cells out of the total number of CD8+T-cells in the skin sample exceeds a cutoff value (e.g., as described above), then the individual can be treated with an anti-PD-1 agent alone (not in combination with an anti-CTLA agent), thereby avoiding the enhanced side effects of that combination of agents.

On the other hand, if the number of CTLA4⁺PD-1⁻CD8+ T-cells out of the total number of CD8+ T-cells in the skin sample is below a cutoff value (e.g., as described above), then the individual can be treated with treatment other than anti-PD-1 therapy, thereby avoiding the risk of side effects from that treatment when it is unlikely to be successful.

In yet another embodiment, individuals who are not predicted to be responsive to anti-PD-1 therapy, i.e., individuals for whom the number of CTLA4⁻PD-1⁺CD8+ T-cells out of the total number of CD8+ T-cells in the skin sample is below a cutoff value (e.g., as described above), can be administered CTLA4⁺PD-1⁺CD8+ T-cells to increase the percentage of CTLA4⁺PD-1⁺CD8+ T-cells thereby increasing the chance that they will respond to anti-PD-1 treatment. In some embodiments, the administered CTLA4⁻PD-1⁺CD8+ T-cells are autologous cells. Thus, in some embodiments, CTLA4⁺PD-1⁻CD8+ T-cells can be purified from the individual, expanded ex vivo, and then introduced back into the individual, thereby boosting the individuals percentage of administered CTLA4⁺PD-1⁻CD8+ T-cells. Expansion of the cells can involve, for example, treating the cells with anti-CD3 and anti-CD28 coated micro beads in the presence of IL-2 to expand the population, e.g., 100-10,000 fold. See, e.g., Tang et al., J Exp Med. 199(11): 1455-1465 (2004). The cells can be administered, for example, intravenously or directly into accessible tumors. In some embodiments, the CTLA4⁺PD-1⁺CD8+ T-cells are initially obtained from the individual by the methods described herein, i.e., obtained from the tissue sample (e.g., a skin sample) treated as described, centrifuged as described, and optionally separated from cellular debris and at least some epithelial cells from the initial sample.

For example, in some embodiments, the individual's percentage of CTLA4⁺PD-1⁻CD8+ T-cells is less than 30% (or 20%) initially and after CTLA4⁺PD-1⁺CD8+ T-cells have been administered to the individual the percentage is increased to more than 30%. Following administration of the CTLA4⁺PD-1⁺CD8+ T-cells , anti-PD-1 therapy can be administered to the individual. Administration of CTLA4⁺PD-1⁺CD8+ T-cells can be as a single dose or can be administered in a series over time to maintain the percentage of CTLA4⁺PD-1⁺CD8+ T-cells during treatment with PD-1 therapy.

### EXAMPLES

The following examples are offered by way of illustration and not by way of limitation.

The ability to reliably and reproducibly assay T cells from human tumor tissue by flow cytometry requires that an adequate number of cells be obtained for analysis. We have significantly modified our protocol to obtain greater than 3.5-fold more T cells per specimen, allowing us to perform flow cytometry on very small amounts of tissue. With this advance, we can now reliably analyze small punch biopsies and core biopsies by flow cytometry. This invention enabled us to discover and quantify the minimum number of tumor-infiltrating PD-1+/CTLA-4+CD8+ T cells needed to achieve a therapeutic response to anti-PD-1 therapy. Based on these results an algorithm was generated to stratify patients for therapy.

### Example 1

Two methods of human skin digestion were assay to determine cell yield and percentages of live, CD3, and CD4 cells obtained.

Materials
1 piece of normal human skin
o abdominal skin, 29 yrs-old female

Skin Digestion Cocktail:

| Enzyme Cocktail | | | | | |
|---|---|---|---|---|---|
| (Digestion Buffer) | Source | Concentration | | Amount | |
| C10 | | | | 10 | mL |
| Collagenase | (Type IV, | 0.8 | mg/mL | 8 | mg |
| | Worthington, | | | | |
| | Cat# LS004186) | | | | |
| DNAse | (from bovine | 20 | μg/mL | 0.2 | mg |
| | pancreas, | | | | |
| | Cat# DN251G) | | | | |

Experimental Design

| Group (for each | | |
|---|---|---|
| skin sample) | Method | Assay |
| 1 | Traditional | Counted on hemocytometer and |
| | | stained for viability, CD45, CD3, |
| | | and CD4 |
| 2 | Shaken + | Counted on hemocytometer and |
| | Slow spin | stained for viability, CD45, CD3, |
| | | and CD4 |

Procedure
1. Fat was be trimmed from sample; sample was not properly shaped for dermatome. Skin was placed on gauze in PBS+Pen/strep at 4 deg C. until ready for processing.
2. Digestion buffer was prepared immediately prior to processing.
3. Skin was partitioned into two portions of equal size/weight. Weight Group 1: 4.0 g Weight Group 2: 3.16 g
4. Slices of skin was minced and placed in 3 mL digestion media in 6 well plates, with one slice minced per well. Group 1 and Group 2 were processed to make an identical number of wells: 6 wells per sample
5. Skin was digested for about 12 hours overnight.
6. Method 1: Skin was mashed through 100 micron filter and washed in C10. Time to process: Equivalent for both methods ˜20 minutes
7. Method 2: Skin was transferred to a 50 mL conical; wells were washed with C10 and wash was transferred to conical tubes. Samples were shaken vigorously in 50 mL conical and then spun 5 min at 300 RPM at 4 deg C. The superficial surface layer containing predominantly epithelial cells was carefully removed and discarded. Supernatant was pipetted through a 100 micron filter into a new 50 mL conical.
8. Cells from each method were be counted.
9. Cells from method 1 and 2 were stained for CD45, CD3, CD4, CD8, and viability, using a standard flow cytometry staining protocol.
10. Cells were analyzed using a multi-parameter flow cytometry and T cell subsets quantified.

Results:

| | Method 1 | Method 2 |
|---|---|---|
| Count on hemocytometer | 4.625 × 10\hat{ }6 | 13.1 × 10\hat{ }6 |
| % Viable cells | 44.8% | 41.4% |
| % CD45+ | 3.28% | 4.53% |
| % CD3 + T cells of CD45 | 25.7% | 32.4% |
| total # of T cells (live | 1.188 × 10\hat{ }6 | 4.244 × 10\hat{ }6 |
| CD3+/CD45+ cells) | | |
| % CD3 + CD4+ T cells of | 81.2% | 72.1% |
| CD3 | | |

Conclusions:

The single density centrifugation method yielded many more cells with a “cleaner” lymphocyte pellet (i.e. fewer epithelial cells) than the comparator filtration-based method.

### Example 2

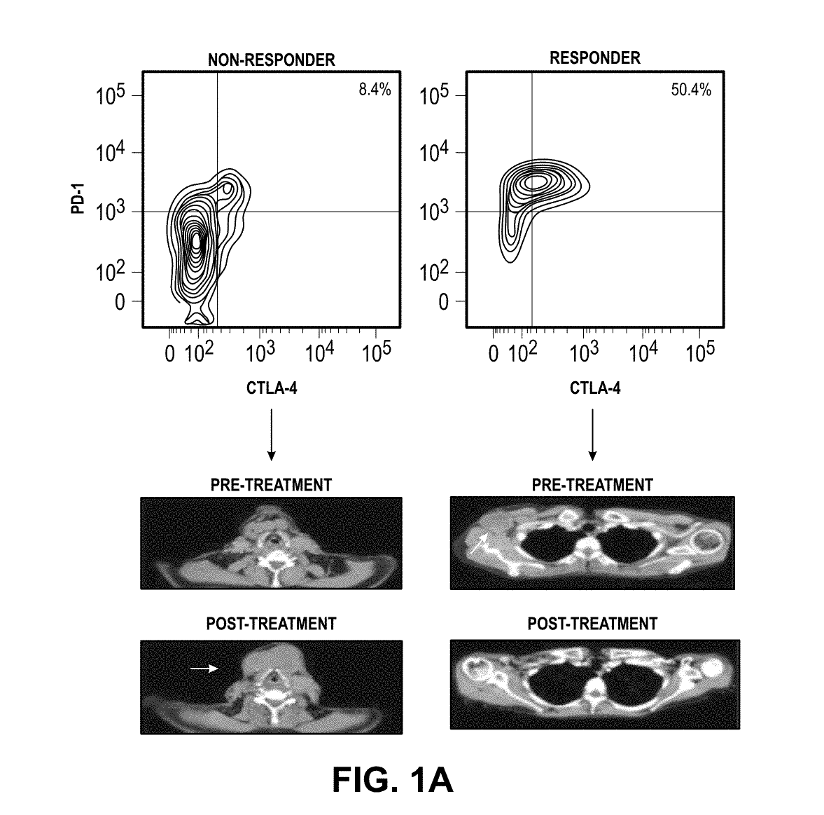

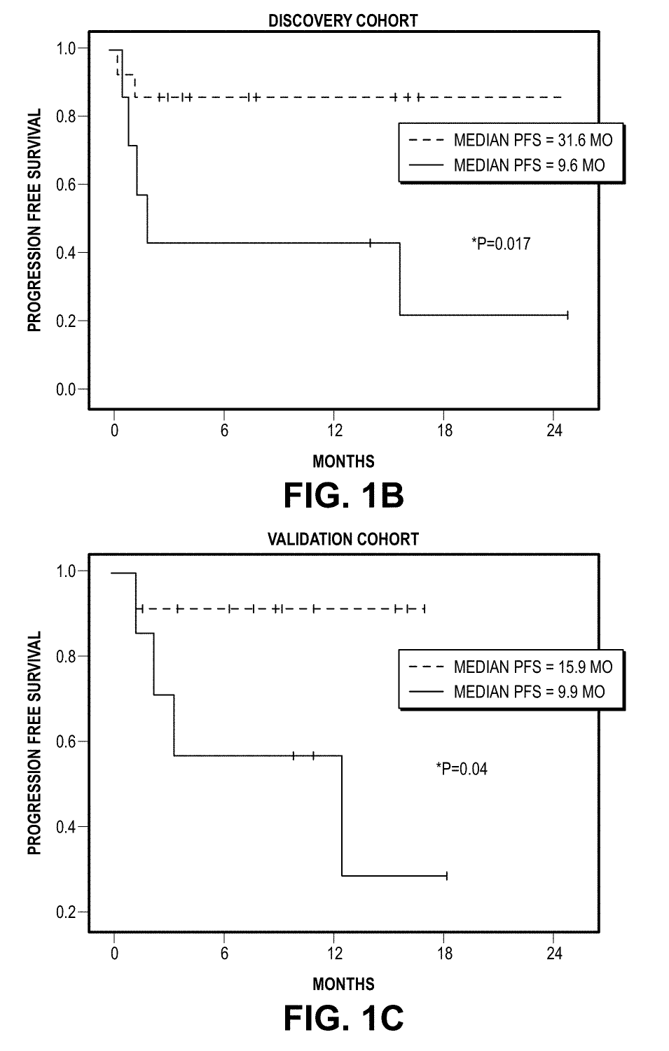

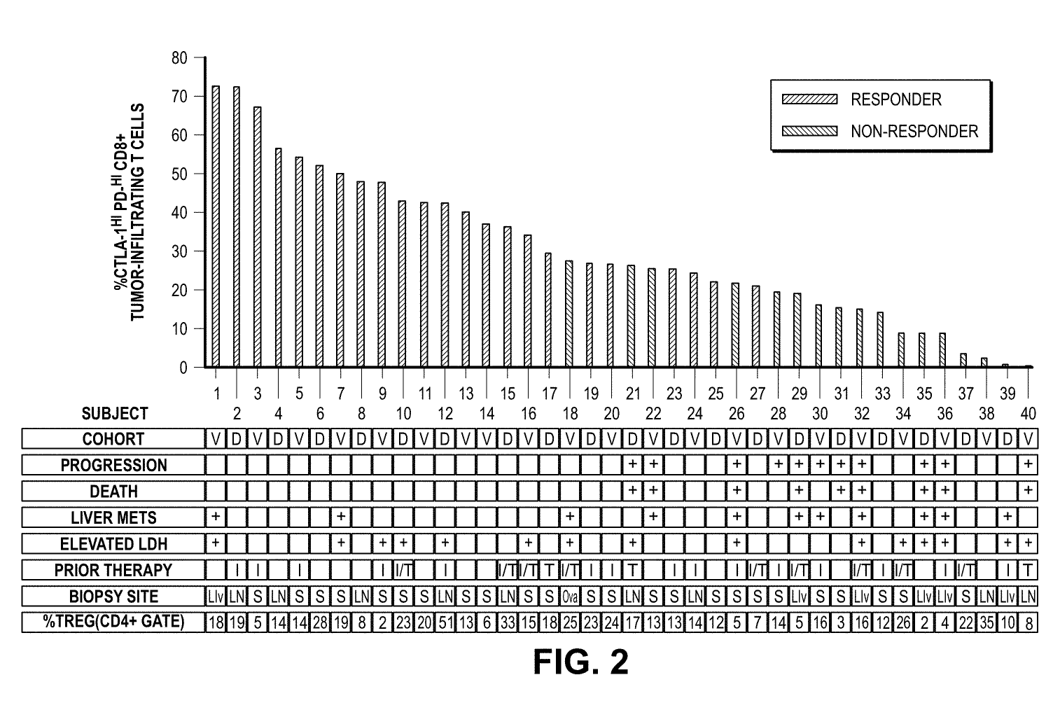

We assessed CD45, CD3, CD4, CD8 and Foxp3 expression to quantify the relative percentages of CD4⁺ effector T cells (CD45⁺CD3⁺CD4⁺Foxp3⁻; Teff cells), CD4⁺ regulatory T cells (CD45⁺CD3⁺CD4⁺Foxp3⁺; Treg cells) and CD8⁺ cytotoxic T cells (CD45⁺CD3⁺CD4⁻CD8⁺; CTLs) infiltrating tumors (Supplemental FIGS. 1A-1B). In addition, we quantified PD-1, PD-L1, CTLA-4 and HLA-DR expression on each of these subsets. Patients were then treated with anti-PD-1 monotherapy and clinical outcome data was collected. Therapeutic response was evaluated using response evaluation criteria in solid tumor malignancies (RECIST v 1.1). Patients with complete or partial responses were considered to be responders and those with stable or progressive disease were considered to be non-responders. Multivariate analysis revealed that CTL expression of CTLA-4 was the single parameter that showed a statistically significant association with achieving a clinical response (Supplemental FIGS. 2A-2C). Further analysis of CTLA-4-expressing CTLs revealed that this subset of CD8⁺ T cells expressed the highest levels of PD-1 (Supplemental FIGS. 2A-2C). Representative flow cytometry plots and clinical images of a non-responder and a responder to anti-PD1 therapy are shown in FIG. 1A. Immunophenotype of a non-responder clearly shows the presence of tumor-infiltrating CD8⁺CTLs; however, these cells express low levels of CTLA-4 and PD-1. In contrast, patients that responded to this treatment had relatively increased percentages of CTLA-4⁺PD-1⁺ cells within the CTL gate.

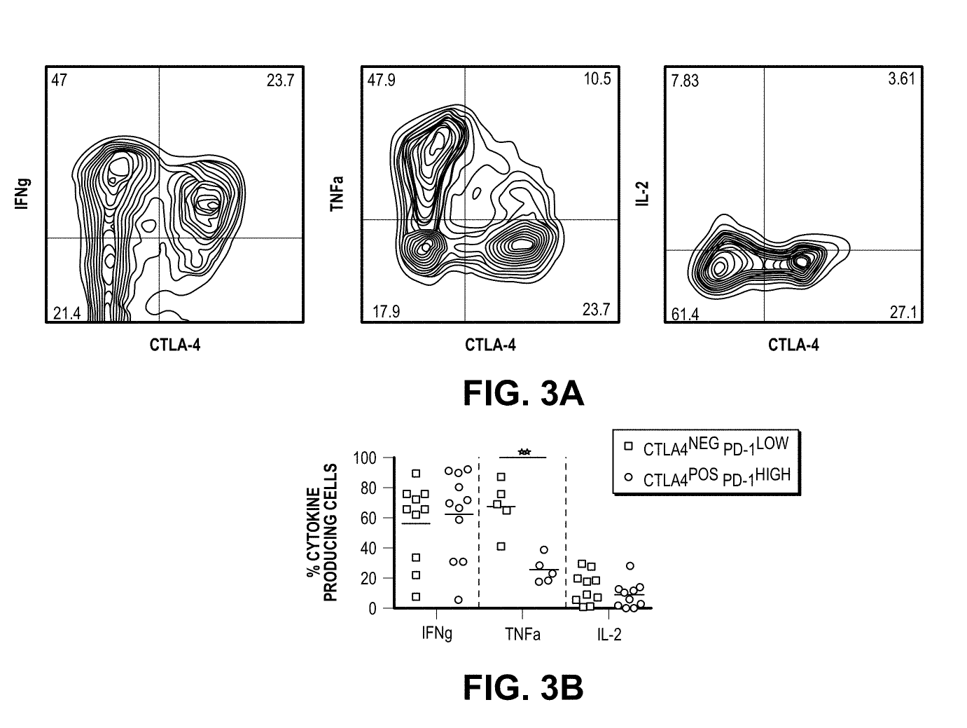

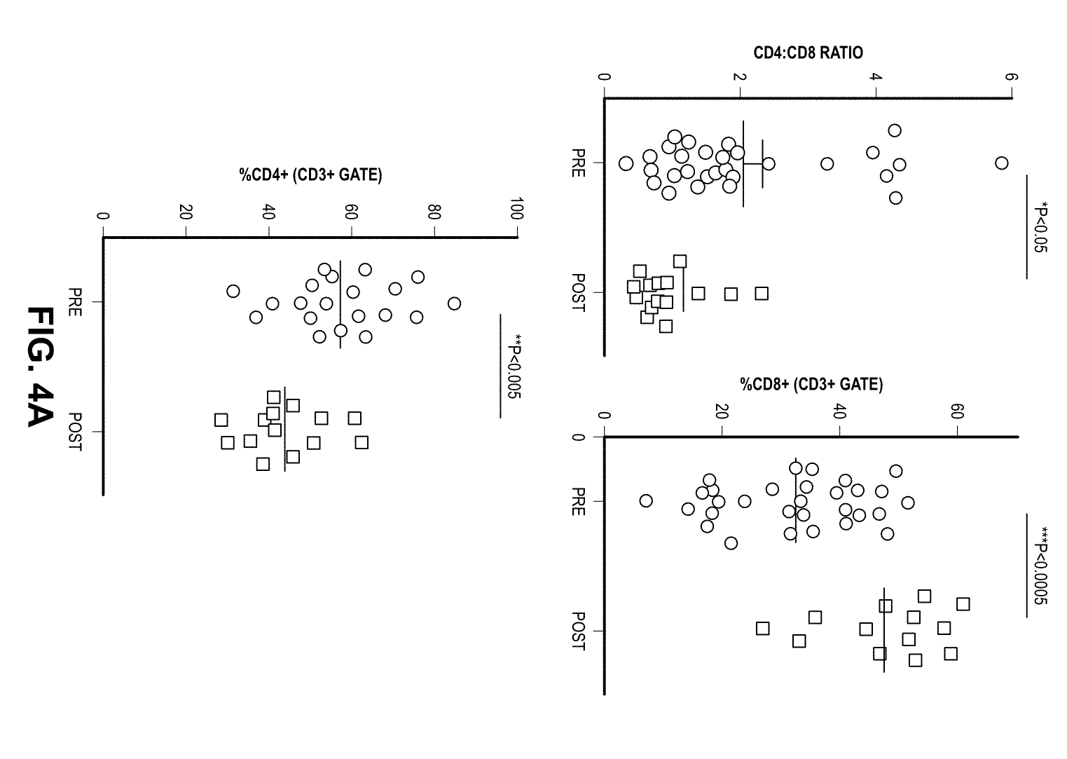

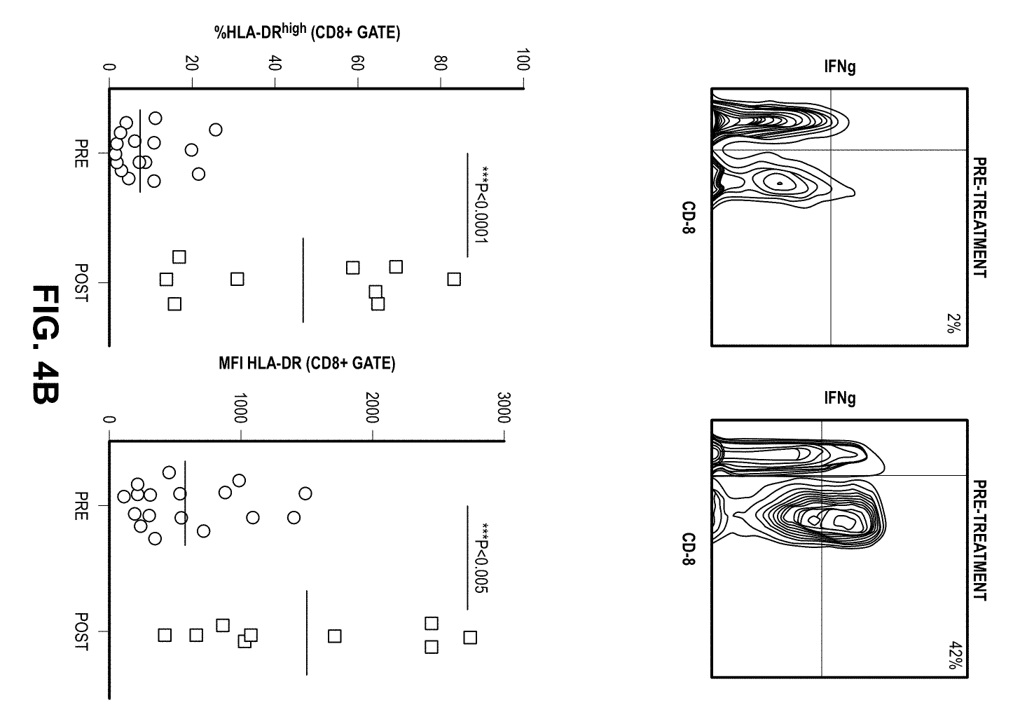

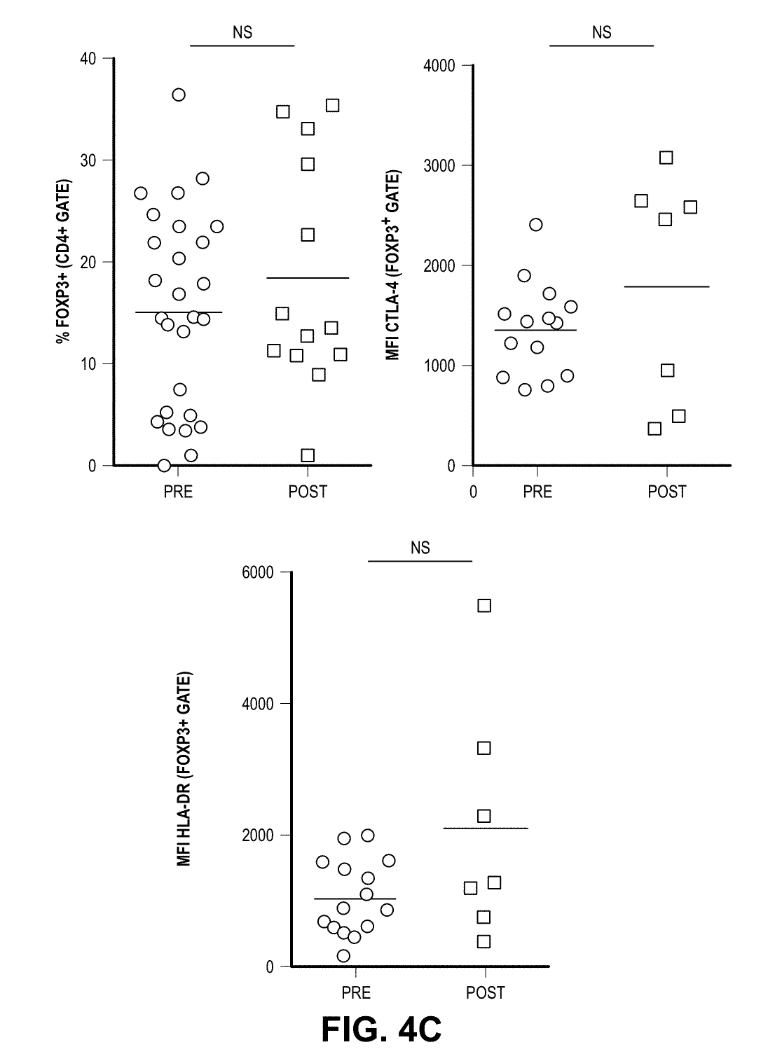

In a discovery cohort of 20 patients, RR and PFS significantly correlated with the relative abundance of CTLA-4⁺PD⁺CD8⁺ tumor-infiltrating CTLs (FIG. 1B). For patients with >20% CTLA-4⁻PD-1⁺CTLs, the PFS was 31.6 months vs. 9.6 months for tumors that contained ≦20% CTLA-4⁺PD-1⁺ cells within the CTL gate (*p=0.017, Log Rank Test). The objective response rate (CR+PR) was 0% (0/6) in the low CTLA-4⁺PD-1⁺CTL group vs. 85.7% (12/14) in the >20% CTLA-4⁺PD-1⁺CTL group. Having identified a potential biomarker for response to anti-PD-1 therapy, we set out to prospectively validate our results in an independent cohort of patients (n=20). In this validation cohort, patients with ≦20% tumor-infiltrating CTLA-4³⁰PD-1⁺CTLs had a PFS of 9.9 months vs. 15.9 months for the high CTLA-4⁺PD-1⁻CTL group (FIG. 1C). The RR was 78.6% for the high CTLA-4⁺PD-1⁻CTL group vs 0% (0/6) for the low CTLA-4³⁰PD-1⁺ group. No objective clinical responses were observed in patients with ≦20% CTLA-4⁺PD-1⁺CTLs infiltrating their tumors (FIG. 2). Percentages of CTLA-4⁺PD-1⁺CTLs was independent of biopsy site or prior therapy (FIG. 2). In addition, response rates were independent of other tumor-infiltrating T cell populations, including Tregs (FIG. 2). Increased proportions of CTLA-4⁺PD-1⁺CTLs also correlated with overall survival (OS); however, this did not achieve statistical significance during the relatively short study period (Supplemental FIG. 3). Clinical response strongly correlated with the abundance of CTLA-4⁺PD-1⁺ double positive cells within the CD8⁺ gate (with a concomitant reduction in CTLA-4⁻PD-1⁻ double negative cells) and only minimally with CTLA-4⁻PD-1⁺ single positive cells (Supplemental FIG. 4). Taken together, our data indicates that the relative abundance of CTLA-4⁻PD-1⁺CTLs infiltrating metastatic melanoma tumors strongly correlates with clinical response to anti-PD-1 therapy.

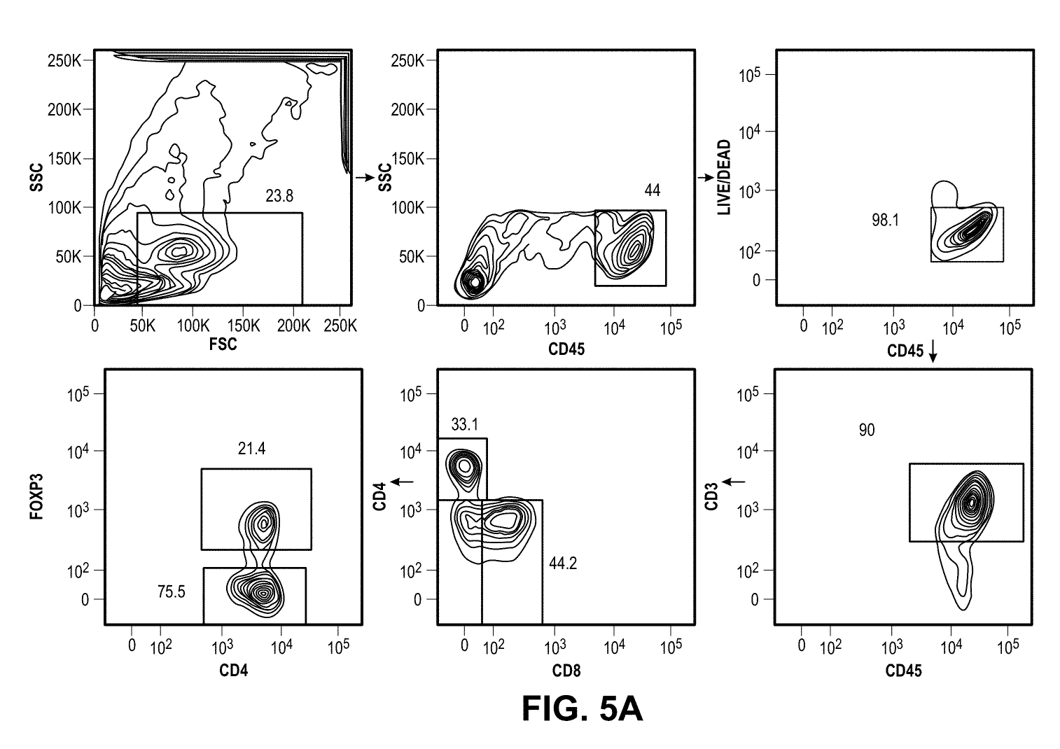

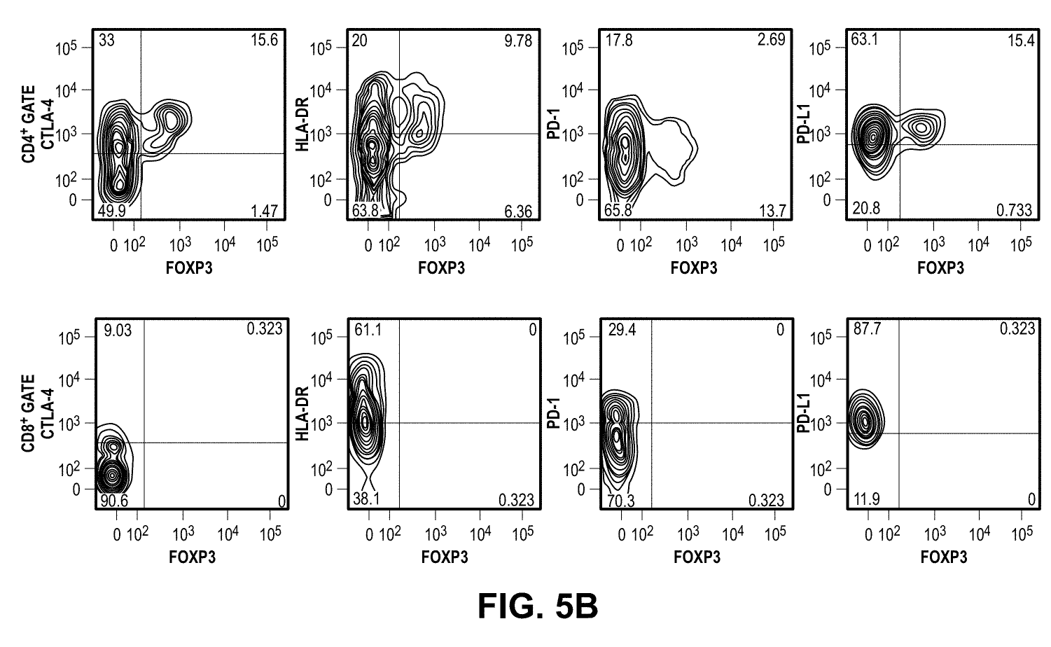

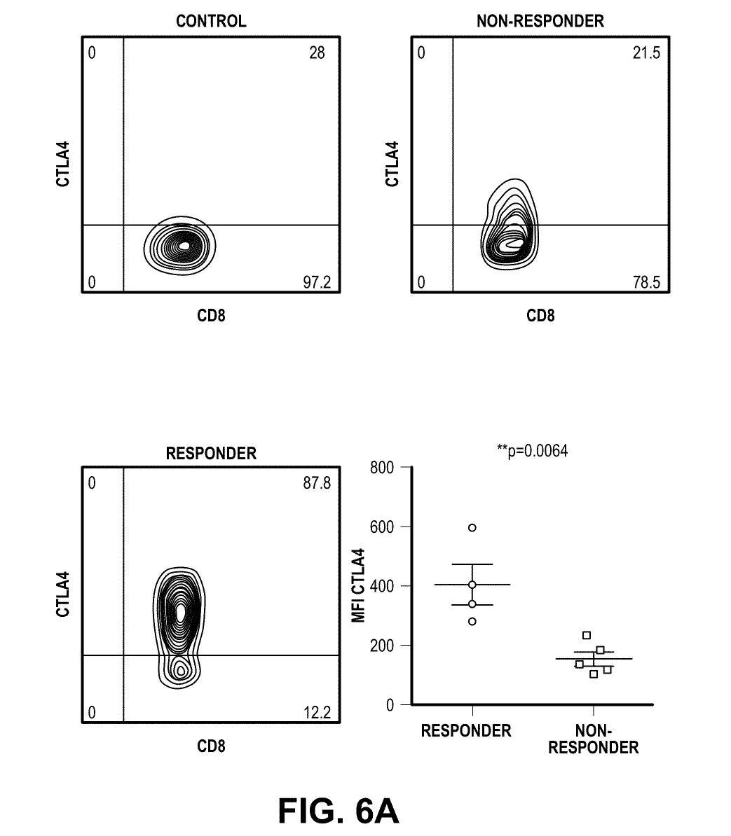

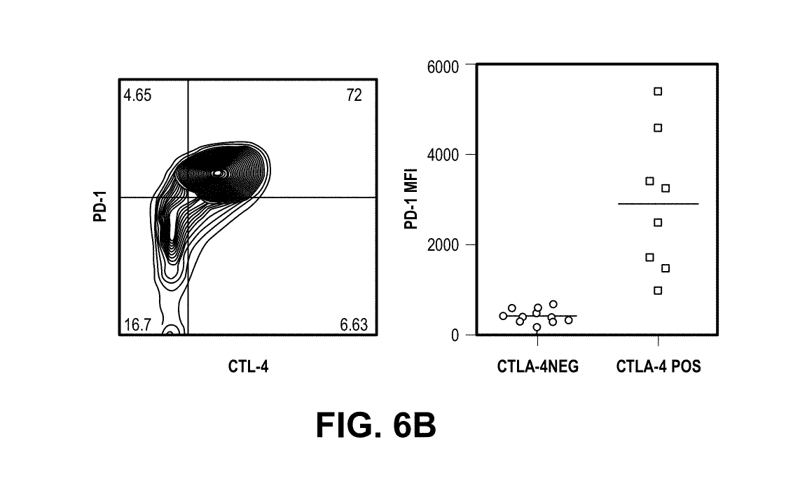

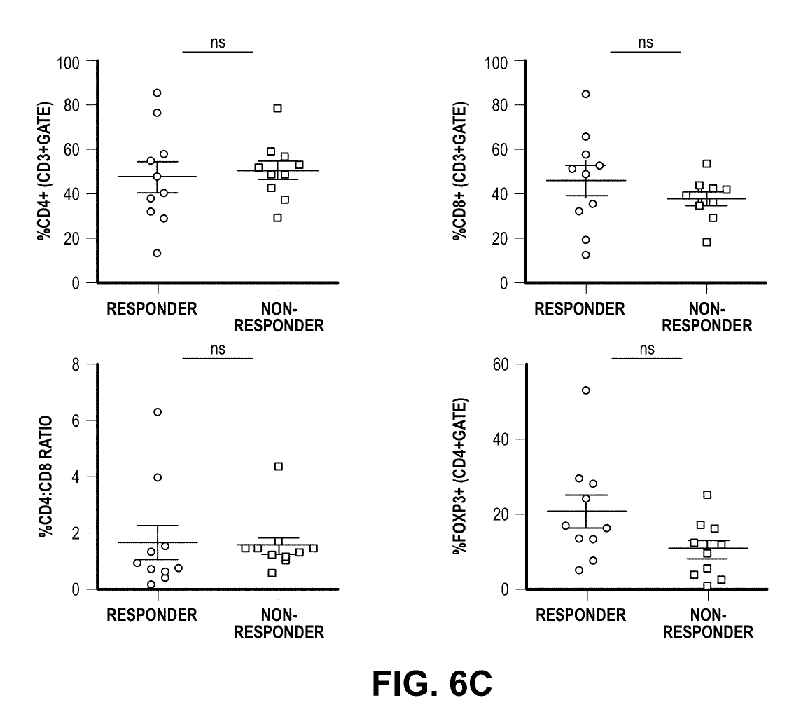

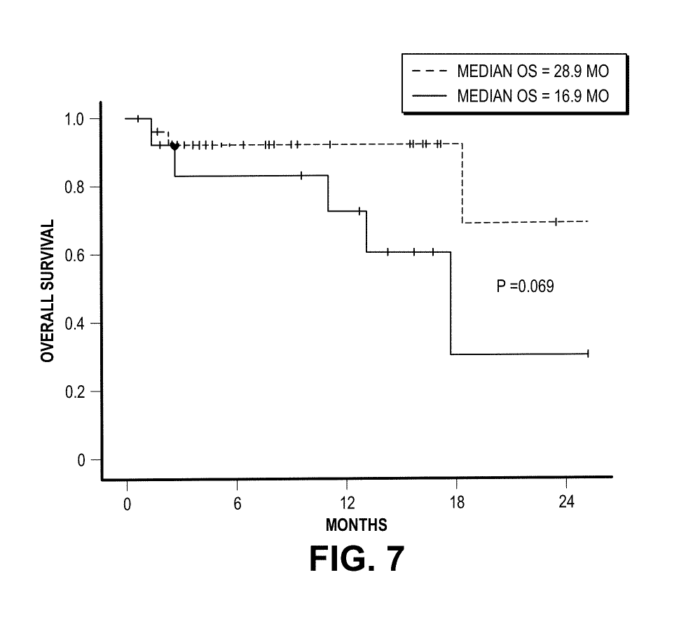

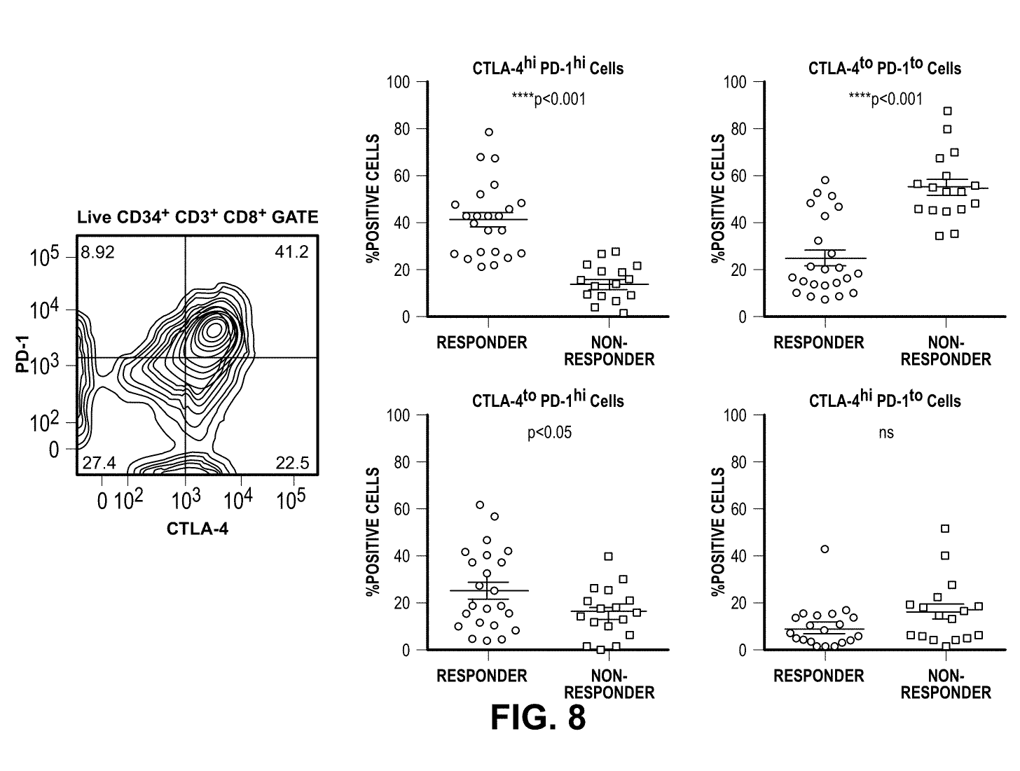

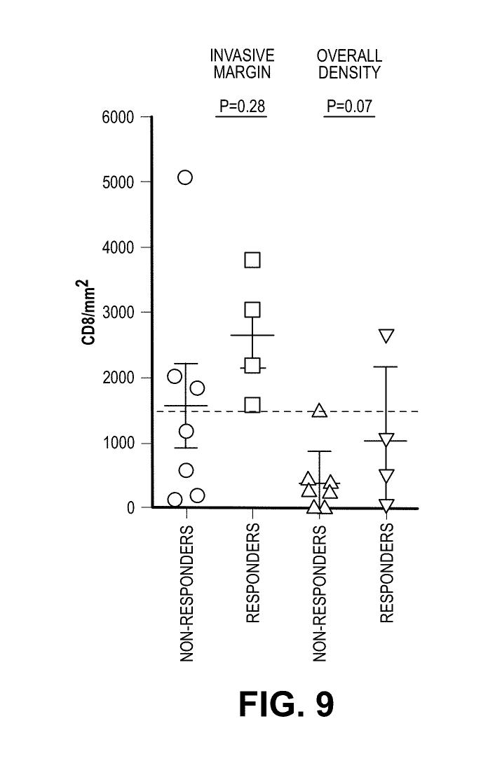

In a previous report, the abundance of CD8⁺ T cells located at the invasive tumor margin predicted response to anti-PD-1 therapy for human melanoma (Tumeh PC et al., Nature 2014; 515(7528):568-571). This assay utilized an immunohistochemical approach to both quantify lymphocytes and discern the tumor invasive margin. We have recently performed this assay on 21 patients with metastatic melanoma (Supplemental FIG. 5). Using 1500 CD8⁺ cells/mm² as a cutoff, in our hands, this assay had a positive predictive value (PPV) of 57% and a negative predictive value (NPV) of 100%. This is comparable to our flow cytometric based assay, which had a PPV of 82% and NPV of 100%. However, using the immunohistochemical approach, 8 of the 21 samples (38%) were not able to be evaluated because the invasive tumor margin could not be reliably discerned. The majority of these samples were smaller biopsy specimens (core biopsies and skin punch biopsies). Because small tumor specimens are the only samples that can be obtained from many patients with skin, liver, lung or bone metastasis, the inability to evaluate these samples is a major limitation of the immunohistochemical assay.

Tumor-infiltrating CTLA-4⁺PD-1⁺CTLs have been shown to contain the majority of tumor-antigen specific T cells (Ahmadzadeh Metal., Blood 2009; 114(8):1537-1544; Gros A et al., J. Clin. Invest. 2014; 124(5):2246-2259). These cells are only detectable in tumors (i.e., not peripheral blood) and have been suggested to have an ‘exhausted’ phenotype (Ahmadzadeh M et al., Blood 2009; 114(8):1537-1544). In order to determine the functional capacity of tumor-infiltrating CTLA-4⁺PD-1⁺CTLs in our patient population, we prepared single cell suspensions from metastatic melanoma tumors and stimulated these cells with PMA/ionomycin. Intracellular cytokine production was then assessed by flow cytometry. When compared to CTLA-4^{neg}PD-1^{low} cells, CTLA-4⁺PD-1⁺CTLs contained equivalent percentages of IFNγ-producing cells; however, CTLA-4⁺PD-1⁺CTLs had a significant reduction in TNFα-producing cells (FIG. 3). Both populations produced very little IL-2 (FIG. 3). In the context of viral infections, a hierarchy of CD8⁺ T cell exhaustion has been described (Freeman G J et al., J. Exp. Med. 2006; 203(10):2223-2227; Wherry E J., Nat. Immunol. 2011; 12(6):492-499). In this model, specific functional properties such as IL-2 and TNFα production are lost first, whereas IFNγ production is more resistant to attenuation. Thus, partially exhausted CD8⁺ T cells have been characterized as cells that are capable of producing IFNγ but lack the ability to produce TNFα and IL-2, a phenotype consistent with tumor-infiltrating CTLA-4⁺PD-1⁺CTLs found in high percentages in patients that responded to anti-PD-1 therapy.

Given that the abundance of CTLA-4⁺PD-1⁺CTLs in metastatic tumors predicts response to anti-PD-1 therapy and that the functional properties of this population is consistent with a partially exhausted T cell phenotype, we set out to determine if treatment with anti-PD-1 reverses CD8⁻ T cell exhaustion in tumors. To test this, we immunophenotyped metastatic lesions during anti-PD-1 therapy and compared these to pre-treatment samples. Treatment with anti-PD-1 resulted in a marked increase in the percentage of tumor-infiltrating CD8⁺ T cells, with a concomitant decline in the percentage of CD4⁺ T cells, resulting in a significant reduction in the CD4:CD8 ratio (FIG. 4). Expression of HLA-DR is a robust activation marker for human CTLs (Ferenczi K et al., J. Autoimmun. 2000; 14(1):63-78; Kestens L et al., AIDS Lond. Engl. 1992; 6(8):793-797; Miller J D et al., Immunity 2008; 28(5):710-722). Thus, we assessed HLA-DR expression on tumor-infiltrating T cells prior to beginning therapy and after the initiation of therapy. When compared to pre-treatment samples, tumor-infiltrating CTLs isolated during therapy had increased percentages of HLA-DR⁺ cells and increased HLA-DR mean fluorescence intensity (FIG. 4). Interestingly, there was no difference in Treg cell percentage or activation (as indicated by either CTLA-4 or HLA-DR expression) between pre-treatment and treatment samples (FIG. 4). These results suggest that treatment with anti-PD-1 preferentially activates CD8⁺CTLs within the tumor microenvironment.

### Discussion

Taken together, our data show that the relative abundance of partially exhausted tumor-infiltrating CTLs correlates with response to anti-PD-1 therapy. In addition, treatment with anti-PD-1 results in increased activation of this population. Thus, the presence of the ‘target’ cell population within tumors is highly predictive of response to therapy. In addition to measuring the abundance of CD8⁺ T cells at the invasive tumor margin (as described above), previous studies have attempted to predict clinical responses by assaying for PD-L1 expression on tumor cells using traditional immunohistochemical approaches (Madore J et al., Pigment Cell Melanoma Res. 2015; 28(3):245-253; Mahoney K M, Atkins M B., Oncol. Williston Park N 2014; 28 Suppl 3:39-48; Pan Z-K et al., J. Thorac. Dis. 2015; 7(3):462-470; Patel S P, Kurzrock R., Mol. Cancer Ther. 2015; 14(4):847-856; Callea M et al., Cancer Immunol. Res. 2015; 3(10):1158-1164). These attempts have yielded variable results. PD-L1 is expressed on many hematopoietic and non-hematopoietic cells within the tumor microenvironment, and in many cases, there is a wide range of staining intensity. Both of these variables make distinctions between relative degrees of tumor PD-L1 expression difficult to discern. In addition, traditional staining is performed on relatively thin sections of tumor and therefore assesses only a small portion of the specimen. In contrast, flow cytometry allows for evaluation of the entire tumor, enabling a more comprehensive assessment of lesions. Flow cytometry is readily performed on small tumor specimens (core and skin punch biopsies), a setting where reliably detecting tumor margins can be challenging. However, it should be noted that flow cytometry of non-hematopoietic tissue is difficult to standardize for clinical use given inherent variability in enzymatic tissue digestion and the requirement for fresh (non-preserved) samples. Future studies focused on developing strategies to robustly quantify partially exhausted CTLs at either the RNA or DNA (i.e., epigenetic) levels using preserved tumor tissue may help to circumvent these issues.

Interestingly, a recent study utilized flow cytometry to show that T cells increase in tumors following PD-1 blockade (Ribas A et al., Cancer Immunol. Res. 2016; 4(3):194-203). These results are consistent with our findings; however, this study was not focused on defining biomarkers that predict response to therapy and thus did not identify the partially exhausted CTL population elucidated in our work. High expression of both CTLA-4 and PD-1 on tumor-infiltrating CTLs was instrumental in identifying our biomarker subset (Supplemental FIGS. 2 & 4), giving mechanistic insight as to how anti-PD-1 therapy results in tumor regression in select groups of patients. Our results are supported by a previous study that found high tumor CTLA-4 expression to be predictive for response to PD-L1 blockade (Herbst R S et al., Nature 2014; 515(7528):563-567).

In our assay, patients that had greater than 20% of total tumor-infiltrating CD8⁻ T cells that co-express high levels of PD-1 and CTLA-4 had an increased likelihood of responding to anti-PD-1 therapy. However, response rates were more variable the closer the percentages were to 20% (FIG. 2). This suggests that there is a subset of patients that have partially exhausted CTLs that hover around the threshold required to achieve a response. Clinical strategies aimed to induce or augment these cells prior to therapy may result in better response rates in these patients. Furthermore, because patients with low percentages of CTLA-4⁺PD-1⁺CTLs are less likely to respond to anti-PD-1 monotherapy, the risk-benefit profile in this patient subset may favor combination therapy with multiple checkpoint inhibitors.

### Methods

### Study Design and Cohorts

Between January 2014 and January 2016, 120 patients with advanced melanoma were treated with the anti-PD-1 antibodies pembrolizumab or nivolumab. Of these patients, 42 had accessible tumors and were amenable to have a research biopsy performed for flow cytometry. Of these, 2 patients were excluded due to inadequate numbers of cells for analysis. Inadequate number of cells was defined as <200 CD8⁺ T cells within the live CD45⁺CD3⁺ gate. Flow cytometric analysis of tumor infiltrating lymphocytes was performed prior to therapy on the 1^{st} discovery cohort of 20 patients (FIG. 1B) and the 2^{nd} validation cohort of 20 patients (FIG. 1C). Pembrolizumab was administered intravenously at 2 or 10 mg/kg every 2 or 3 weeks and Nivolumab was administered at 3 mg/kg every 2 weeks. Patients underwent a core biopsy with a 18 g core or punch biopsy with a 4 mm punch following sterile precautions.

### Treatment Outcome Groups and Efficacy Analysis

Two treatment outcome groups, “Responders” and “Non-Responders”, were defined using radiologic imaging following anti-PD-1 treatment. Responders included patients with tumor target lesions that met RECIST 1.1 criteria for complete response (>99% reduction in the target lesions) or partial response (>30% reduction in target lesions). Non-responders included patients with tumor target lesions that met RECIST 1.1 criteria for progression (>20% increase in the target lesions) or stable disease (<30% reduction or <20% increase in tumor target lesions). Available efficacy and immunological data as of January 2016, was included in all the analyses. The efficacy analysis included two endpoints: (1) best overall response was defined as the best tumor response from the start of treatment to the time of disease progression or death; and (2) progression-free survival (PFS) was defined as the interval between the date of enrollment and the date of progression or death (or the last date of clinic visit where the patient was known not to have had radiological or clinical progression). Overall survival was calculated as time from date of enrollment to the time of death or to the last known date that the patient was known to be alive. Best overall response was determined from investigator-reported data according to RECIST 1.1 criteria. The Rosenblum laboratory team was blinded to all clinical data (including treatment response) throughout all studies.

### Tumor Sample Procurement

Informed consent was obtained for all patients for biopsy of a metastatic lesion within 30 days of starting treatment. Biopsy collection and analyses were approved by the UCSF Institutional Review Board protocol 13-12246. Tumor biopsies were done with either 16 or 18 gauge needles or 4 mm punch tools.

### Flow Cytometric Analyses

Multi-parameter flow cytometry was performed on pre-treatment and treatment samples obtained from metastatic tumors as previously described (Sanchez Rodriguez R et al., J. Clin. Invest. 2014; 124(3):1027-1036). Freshly isolated samples were minced and digested overnight with buffer consisting of Collagenase Type 4 (Worthington 4188), DNAse (Sigma DN25-1G), 10% FBS, and 1% HEPES and 1% Penicillin/Streptavidin in RPMI medium. Single cell suspensions were double filtered, centrifuged, and counted. For intracellular cytokine analysis, digested tumor cell suspensions were stimulated with PMA/ionomycin for 4 hours as previously described (Sanchez Rodriguez Ret al., J. Clin. Invest. 2014; 124(3):1027-1036). Approximately 2×10⁶ cells were stained with multiple fluorochrome-conjugated monoclonal antibodies. The following antibodies were used (eBioscience, unless otherwise stated): anti-hCD3 (UCHT1), anti-hCD8 (RPA-T8), anti-hCD45 (HI30), anti-CD4 (SK3), anti-Foxp3 (PCH101), anti-hCTLA-4 (14D3), anti-PD-1 (EH12.2H7; Biolegend), anti-HLA-DR (LN3), anti-PD-L1 (MIE1) and LIVE/DEAD Fixable Aqua Dead Cell Stain (Life Technologies). Data was acquired by an LSRFortessa (BD Bioscienes) and analyzed using FlowJo software (Tree Star, Inc.).

### Flow Cytometry Standardization and Gating Strategy

All the samples were fresh and acquired by the Fortessa at different time points. To standardize voltages over time, Sphero Ultra Rainbow beads (Spherotech) were used to calibrate and normalize to baseline intensity. Gates were determined using both isotype control antibody staining and an internal negative control cell population (i.e., PD-1 and CTLA-4 expression on CD3⁻ cells).

### Statistical Analysis

Progression-free and overall survival curves were constructed with the Kaplan Meier method and compared with the Maentel-Henzel Log-Rank test using SPSS V23 (IBM Corp., Armonk, N.Y.). Progression was recoreded on the date of scans showing progression or on the date clinical progression or death was noted. All tests were 2-sided with p-values <0.05 considered statistically significant. Patients with tumors <20% CTLA-4⁺PD-1⁺ were noted to have infrequent responses in intial testing and this threshold was used in the discovery and validation cohorts. For flow cytometric data, significance was assessed using the unpaired 2 tailed Student's t test. In all figures quantifying flow cytometric data, the mean value is visually depicted. P values correlate with significance symbols as follows: ns p>0.05, *p<0.05, **p<0.01, ***p<0.001, ****p<0.0001. A p value of less than 0.05 was considered significant. Statistical analysis was done using GraphPad Prism software (GraphPad).

### Study Approval

Melanoma tumors were biopsied following informed consent under the UCSF Committee on Human Research Protocol #138510.

### Example 3

As an alternative method of the single density centrifugation method, the following protocol was followed:

Subcutaneous fat and hair were removed, and skin containing some digestion media (400 U/ml Collagenase IV, 200 ug/ml DNAse, 1% penicillin/streptavidin in RPMI medium) was minced finely with dissection scissors and mixed in a 15 ml conical with 1.5 ml of digestion buffer per 14 mm² punch biopsy.

Samples were then placed on agitator for 2.5 hours at 37° C., harvested with wash buffer (2% FBS, 1% penicillin/streptavidin in RPMI medium), and shaken by hand about 30 seconds. If skin was originally larger than 1 cm², samples were then centrifuged at 17 rcf for 5 minutes to form a gradient between epithelial cells, lymphocytes and debris. Epithelial cells will remain near the top of the suspension, lymphocytes will stay in the supernatant, and debris will pellet to the bottom. Therefore, the middle layer of suspended cells was harvested. Samples were then filtered once through a 100 um filter, centrifuged, and counted.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entireties for all purposes.

What is claimed is:

1. A method of determining a percentage of CD8⁺ T-cells being CTLA4⁺PD-1⁺ in a skin sample comprising a metastatic lesion from a human individual, the method comprising,
digesting the skin sample in a digestion medium comprising a protease, a nuclease, or both a protease and a nuclease to generate a digested skin sample;
centrifuging the digested skin sample in a single density solution to separate epithelial cells, T-cells, and insoluble debris;
extracting the T-cells from the single density solution, thereby separating the lymphocytes from at least some of the epithelial cells and insoluble debris;
and contacting the extracted T-cells with: (a) an anti-CD8 antibody, (b) an anti-CTL4 antibody, and (c) an anti-PD-1 antibody and calculating the percentage of CD8⁺ T-cells being CTLA4⁺PD-1⁺ based upon the amount of anti-CD8 antibody, anti-CTL4 antibody, and anti-PD-1 antibody that bind the extracted T-cells.

2. The method of claim 1, further comprising after the digesting and before the centrifuging, agitating the digested skin sample in a mixing solution.

3. The method of claim 2, wherein the mixing solution comprises serum.

4. The method of claim 3, wherein the mixing solution comprises fetal bovine serum, RPMI medium, and one or more antibiotic.

5. The method of claim 1, wherein the protease is a collagenase and the nuclease is a DNAse.

6. The method of claim 1, wherein the metastatic lesion is a melanoma metastatic lesion.

7. The method of claim 1, further comprising comparing the percentage to a cut-off value,
wherein if the percentage exceeds the cut-off value, the method further comprises administering a therapeutic amount of a PD-1 antibody to the individual.

8. The method of claim 7, wherein the cut-off value is between 10-40% CTLA4^{hi}PD-1^{hi}CD8^{+T-}cells.

9. A method of converting a human individual having a metastatic lesion in skin and who is non-responsive to anti-PD-1 therapy into a responder to anti-PD-1 therapy, the method comprising
obtaining CTLA4⁺PD-1⁺CD8⁺ cells from the individual;
expanding the CTLA4⁺PD-1⁺CD8⁺ cells ex vivo; and
introducing the expanded cells into the individual, thereby increasing the responsiveness of the individual to anti-PD-1 treatment.

10. The method of claim 9, wherein the individual has melanoma.

11. The method of claim 9, further comprising administering an anti-PD-1 molecule to the individual after the introducing.

12. The method of claim 11, wherein the anti-PD-1 molecule is an antibody.

13. A method of detecting a cell-surface marker in T-cells from a skin sample, the method comprising,
digesting the skin sample in a digestion medium comprising collagenase and DNAse to generate a digested skin sample;
centrifuging the mixed digested skin sample in a single density solution to separate epithelial cells, T-cells, and insoluble debris;
extracting the T-cells from the single density solution, thereby separating the lymphocytes from at least some of the epithelial cells and insoluble debris;
and detecting one or more cell surface markers on the extracted T-cells with an antibody, thereby detecting a cell-surface marker in T-cells from a skin sample.

14. The method of claim 13, wherein the protease is a collagenase and the nuclease is a DNAse.

15. The method of claim 13, further comprising after the digesting and before the centrifuging, agitating the digested skin sample in a mixing solution.

16. The method of claim 15, wherein the mixing solution comprises serum.

17. The method of claim 16, wherein the mixing solution comprises fetal bovine serum, RPMI medium, and one or more antibiotic.
